(19) United States
(12) Patent Application Publication
(10) Pub. No.: US 2025/0342958 A1
(43) Pub. Date: Nov. 6, 2025

(54) CANCER TYPE PREDICTION MODEL ESTABLISHMENT SYSTEM, CANCER TYPE PREDICTION SYSTEM AND METHOD USING THE SAME

(71) Applicants: Inventec Easy Doctor Corporation, Shanghai (CN); Inventec Appliances (Pudong) Corporation, Shanghai (CN); Inventec Appliances (Shanghai) Co.,Ltd., Shanghai (CN); Inventec Appliances Corp., New Taipei City (TW)

(72) Inventor: Chih-Peng LIN, New Taipei City (TW)

(21) Appl. No.: 18/703,472

(22) PCT Filed: Mar. 8, 2023
(86) PCT No.: PCT/CN2023/080283

§ 371 Date: Jul. 20, 2025

(51) Int. Cl. G16H 50/20

(52) U.S. Cl. CPC G16H 50/20

(57) ABSTRACT

A cancer type prediction model establishment system includes a first-level learning model establishment unit and a second-level first learning model establishment unit. The first-level learning model establishment unit is for establishing a first-level learning model according to a first CNV, a first sample cancer type and a first gender of each first learning sample by using a machine learning technology; and a second gender and a second copy number variation of each second learning sample are taken as an input of the first-level learning model, so that the first-level learning model outputs a first output cancer type of each second learning sample. The second-level first learning model establishment unit is for establishing a second-level first learning model according to the first output cancer types and a second sample cancer type of each second learning sample by using machine learning technology.

This application is the 35 U.S.C. § 371 national stage of PCT application PCT/CN2023/080283, filed Mar. 8, 2023, the disclosure of which is hereby incorporated by reference.

### FIELD OF THE INVENTION

The invention relates to a cancer type prediction model establishment system, a cancer type prediction system and a method using the same.

### BACKGROUND OF THE INVENTION

Cancer may occur in many organs of the human body, such as liver, kidney, gastrointestinal tract, brain, etc. Cancer could be diagnosed and treated early through regular physical examinations to improve the cure effect. Therefore, how to predict or diagnose cancer types is one of the goals of those skilled in the art.

### SUMMARY OF THE INVENTION

The present disclosure proposes a cancer type prediction model establishment system, a cancer type prediction system and a cancer type prediction method using the same, which are capable of improving the aforementioned conventional problems.

In an embodiment of the invention, a cancer type prediction system is provided. The cancer type prediction system includes a first-level learning model establishment unit and a second-level first learning model establishment unit. The first-level learning model establishment unit is configured to establish a first-level learning model, by using a machine learning technique, according to a first CNV (copy number variation), a first sample cancer type and a first gender of each of a plurality of first learning sample; and use a second gender and a second CNV of each of a plurality of second learning sample as an input of the first-level learning model, so that the first-level learning model outputs a plurality of first output cancer types of each second learning sample. The second-level first learning model establishment unit is configured to establish a second-level first learning model, by using the machine learning technique, according to the first output cancer types and a second sample cancer type of each of the second learning samples.

In another embodiment of the invention, a cancer type prediction system is provided. The cancer type prediction system includes a storage unit and a prediction unit. The storage unit is configured to store the first-level learning model and the second-level first learning model as described aforementioned above. The prediction unit is configured to obtain a plurality of first prediction cancer types of the to-be-tested sample by inputting a sample CNV and a sample gender of a to-be-tested sample to the first-level learning model; determine whether a sample gender of the to-be-tested sample is the second gender; and when the sample gender of the to-be-tested sample is the second gender, obtain a second prediction cancer type of the to-be-tested sample by inputting the first prediction cancer type of the to-be-tested sample to the second-level first learning model.

In another embodiment of the invention, an establishing method for a cancer type prediction model is provided. The establishing method includes the following steps: establishing a first-level learning model, by using a machine learning technique, according to a first CNV, a first sample cancer type and a first gender of each of a plurality of first learning sample; using a second gender and a second CNV of each of a plurality of second learning sample as an input of the first-level learning model, so that the first-level learning model outputs a plurality of first output cancer types of each second learning sample; and establishing a second-level first learning model, by using the machine learning technique, according to the first output cancer types and a second sample cancer type of each of the second learning samples.

In another embodiment of the invention, a cancer type prediction method is provided. The cancer type prediction method includes the following steps: obtaining a first prediction cancer type of the to-be-tested sample by inputting a sample CNV and a sample gender of a to-be-tested sample to a first-level learning model as described aforementioned above; determining whether a sample gender of the to-be-tested sample is the second gender; and when the sample gender of the to-be-tested sample is the second gender, obtaining a second prediction cancer type of the to-be-tested sample by inputting the first prediction cancer type of the to-be-tested sample to the second-level first learning model as described aforementioned above. Wherein the second gender is different from the third gender.

### BRIEF DESCRIPTION OF THE DRAWINGS

The above objects and advantages of the invention will become more readily apparent to those ordinarily skilled in the art after reviewing the following detailed description and accompanying drawings, in which:

FIG. 1 shows a functional block diagram of a cancer type prediction model establishment system according to an embodiment of the present disclosure;

FIG. 2 shows a functional block diagram of a cancer type prediction system according to an embodiment of the present invention;

FIG. 3 shows a flowchart of an establishing method for a cancer type prediction model in the cancer type prediction model establishment system in FIG. 1; and

FIG. 4 shows a flow chart of the cancer type prediction method of the cancer type prediction system in FIG. 2.

### DETAILED DESCRIPTION OF PREFERRED EMBODIMENTS

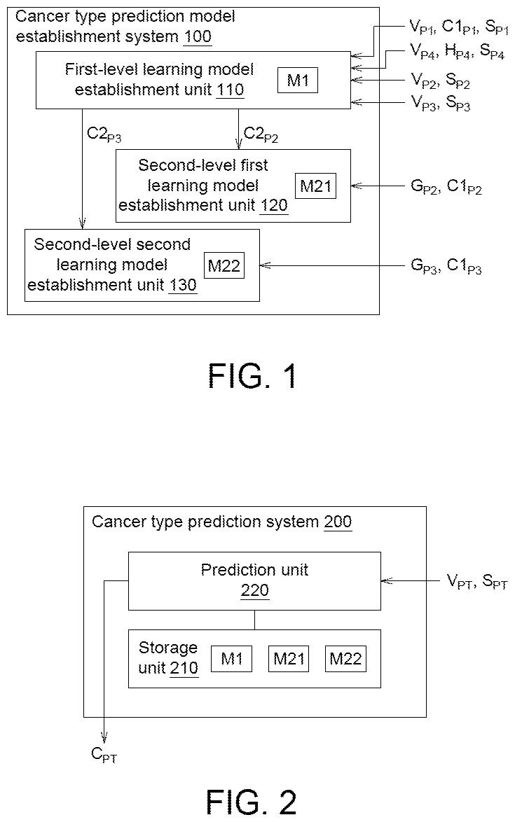

Referring to FIG. 1, FIG. 1 shows a functional block diagram of a cancer type prediction model establishment system 100 according to an embodiment of the present disclosure. The cancer type prediction model establishment system 100 includes a first-level learning model establishment unit 110, a second-level first learning model establishment unit 120 and a second-level second learning model establishment unit 130. The first-level learning model establishment unit 110, the second-level first learning model establishment unit 120 and/or the second-level second learning model establishment unit 130 are, for example, physical circuits formed by at least one semiconductor manufacturing process. In an embodiment, at least two of the first-level learning model establishment unit 110, the second-level first learning model establishment unit 120 and the second-level second learning model establishment unit 130 could be integrated into a single unit. In an embodiment, at least one of the first-level learning model establishment unit 110, the second-level first learning model establishment unit 120 and the second-level second learning model establishment unit 130 could be integrated into a controller or in a processor.

As shown in FIG. 1, the first-level learning model establishment unit 110 is configured to: (1). establish a first-level learning model M1, by using a machine learning technique, according to a first CNV (copy number variation) V_{P1}, a first sample cancer type (the cancer type of the person to whom the learning sample belongs) C1_{P1} and a first gender (the gender of the person to whom the learning sample belongs) S_{P1} of each of a plurality of first learning sample P1 (not illustrated); (2). use a second gender S_{P2} and a second CNV V_{P2} of each of a plurality of second learning sample P2 (not illustrated) as an input of the first-level learning model M1, so that the first-level learning model M1 outputs a plurality of first output cancer types C2_{P2} of each second learning sample P2. The second-level first learning model establishment unit 120 is configured to establish a second-level first learning model M21, by using the machine learning technique, according to the first output cancer types C2_{P2} and at least one second sample cancer type C1_{P2} of each of the second learning samples P2. In the present embodiment, the first-level learning model M1 is a mixed learning model (for example, not limited to gender), accordingly it could avoid the learning (or training) error caused by some cancer types with a small number of samples.

As shown in FIG. 1, in an embodiment, the first-level learning model establishment unit 110 is further configured to establish the first-level learning model M1, by using machine learning technology, according to a fourth CNV V_{P4}, a health category H_{P4} and a fourth gender S_{P4} of each of a plurality of healthy samples P4 (not illustrated). As a result, the established first-level learning model M1 further includes the health category H_{P4}, and it could determine a to-be-tested sample belonging to the health category H_{P4}. The fourth gender S_{P4} is not limited to male or female. The copy number of the genome segment of the healthy sample P4 is not abnormally increased or decreased (without gene mutation), that is, its fourth CNV V_{P4} is normal.

In addition, the second-level first learning model M21 could be established according to not only the first output cancer types C2_{P2} and the second sample cancer type C1_{P2}, but also other sample information, such as the age of the learning sample. For example, as shown in FIG. 1, the second-level first learning model establishment unit 120 could establish the second-level first learning model M21 according to the first output cancer types C2_{P2}, the second sample cancer type C1_{P2} and the age Gp2 of each second learning sample P2.

As shown in FIG. 1, other model could be established according to other genders except that the second-level first learning model M21 could be established according to the second gender S_{P2}. For example, the first-level learning model establishment unit 110 is further configured to: use a third gender S_{P3} and a third CNV V_{P3} of each of a plurality of third learning sample P3 (not illustrated) as an input of the first-level learning model, so that the first-level learning model M1 outputs a plurality of second output cancer types C2_{P3} of each third learning sample P3. The second-level second learning model establishment unit 130 is configured to establish the second-level second learning model M22, by using machine learning technology, according to the second output cancer types C2_{P3} and a third sample cancer type C1p3 of each third learning sample P3.

In addition, the second-level second learning model M22 could be established according to not only the second output cancer types C2_{P3}, but also other sample information, such as the age of the learning sample. For example, as shown in FIG. 1, the second-level second learning model establishment unit 130 could establish the second-level second learning model M22 according to the age of the second output cancer types C2_{P3}, the third sample cancer type C1p3 and the age Gpsof each third learning sample P3.

In addition, the output cancer type (for example, the first output cancer types C2_{P2} and/or the second output cancer types C2_{P3}) herein could be expressed in a probability form, for example. Table 1 below lists the probabilities of a plurality of the first output cancer types C2_{P2} outputted by the first-level learning model M1. Table 1 only takes 5 learning samples as an example, but the number of the learning samples could be more than five. As shown in Table 1 below, different numbers represent different learning samples. These first output cancer types C2_{P2} are different cancer types, such as liver cancer, prostate cancer, breast cancer, etc. In case of the sample number #1 as an example, the probability of the liver cancer (the first output cancer types C2_{P2}) is 9.958%, the probability of the prostate cancer is 77.335%, and the probability of the breast cancer is 0.011%. Others first output cancer types C2_{P2} are not listed, but each of them has a probability value. The numerical values in Table 1 are merely for example, and it is not intended to limit the embodiments of the present invention. The interpretation methods for other sample numbers are similar to number #1, and the 5 similarities will not be repeated here. The second-level first learning model establishment unit 120 could establish the second-level first learning model M21 according to the probability information in Table 1. In addition, the second-level second learning model establishment unit 130 could also establish the second-level second learning model M22 according to the probability information (the second output cancer types C2_{P3}) similar to Table 1.

TABLE 1

Category

| | liver | prostate | breast | | |
|---|---|---|---|---|---|
| sample | cancer | cancer | cancer | . . . | health |
| #1 | 0.09958 | 0.77335 | 0.000111 | . . . | 0.0002 |
| #2 | 0.01061 | 0.20688 | 0.122995 | . . . | 0.0001 |
| #3 | 0.12278 | 0.09811 | 0.057845 | . . . | 0.0001 |
| #4 | 0.007043 | 0.36396 | 0.050012 | . . . | 0.0001 |
| #5 | 0.007040 | 0.02467 | 0.000051 | . . . | 0.0001 |

In addition, the machine learning technique in this disclosure is, for example, a support vector machine (SVM). However, the embodiment of the present invention does not limit the type of machine learning technology. As long as it could learn the input information required in this disclosure and could establish a cancer type learning model, it could be used as the application of the machine learning technology in this disclosure.

In addition, the leaning sample herein is, for example, circulating tumor cells (CTC). Circulating tumor cells refer to tumor cells that break away from tumor tissue and enter the blood. Circulating tumor cells from different organs tend to carry specific types of gene mutations. Thus, by detecting the specific gene mutation types carried by circulating tumor cells, the organ (cancer types) of the circulating tumor cells could be it could be inferred. The use of the circulating tumor cells as samples has the following advantages: (1). examination could be done by drawing blood (no physical section or radiation imaging is required), and the cost and risk are low; (2). it is suitable for long-term monitoring for cancer recurrence; (3). overcoming tumor heterogeneity; (4). early prediction for metastasis; and (5). rapid response to current tumor status.

Furthermore, copy number variation (CNV) is a phenomenon in which portions of the genome are repeated, and the number of repetitions in the genome varies between individuals. Furthermore, copy number variation is a duplication event or a deletion event that affects a large number of Alkali-based pairs. Approximately two-thirds of the whole human genome may consist repeatedly, and 4.8% to 9.5% of the human genome could be classified as copy number variation. The CNV abnormalities are determined when the copy number of the genomic segment increases.

In addition, the leaning samples in the present disclosure are taken from, for example, the Cancer Genome Atlas (TCGA), which records the name of the cancer type, the age, the CNV, the gender, etc. of each sample. TCGA collects about 34 names of the cancer types. In an embodiment, the 34 names of the cancer types could be summarized or integrated into, for example, 12 names of the cancer types or less, such as brain cancer, esophageal cancer, lung cancer, kidney cancer, and male cancer (names of the cancer types include, for example, testicular cancer, prostate cancer, etc.) women's cancer ((names of the cancer types include, for example, cervical cancer, uterine cancer, endometrial cancer, etc.), liver cancer, bladder cancer, anterior mediastinum cancer (names of the cancer types include, for example, thyroid cancer, thymus cancer), head and neck cancer, breast cancer and/or gastrointestinal cancer (names of the cancer types include, for example, colorectal cancer, rectal cancer, pancreatic cancer, gastric cancer, etc.), etc. In addition, the first gender S_{P1} herein is not limited to male or female. For example, the first genders S_{P1} of these persons to whom the first learning samples P1 belongs could be a combination of male and female. The second gender is different from the third gender. The second gender S_{P2} and the third gender S_{P3} are limited to one of male and female. For example, the second genders S_{P2} of these persons to whom the second learning samples P2 belongs could be male, and the third genders S_{P3} of these persons to whom the third learning samples P3 belongs could be female. Alternatively, the second genders S_{P2} of these persons to whom the second learning samples P2 belongs could be female, and the third genders S_{P3} of these persons to whom the third learning samples P3 belongs could be male.

Referring to the following Table 2, in the present embodiment, in case of the sum of the sample number for the first-level learning model M1, the sample number for the second-level first learning model M21, the sample number for the second-level second learning model M22 and the sample number for verification being 100%, the sample number for learning model accounts for 80%, and the sample number for verification accounts for 20%. in case of the sum of the sample number for the first-level learning model M1, the sample number for the second-level first learning model M21 and the sample number for the second-level second learning model M22 being 100%, the sample number for the first-level learning model M1 accounts for 80%, and the sum of the sample number for the second-level first learning model M21 and the sample number for the second-level second learning model M22 accounts for 20%. These samples could be obtained from TCGA, hospitals and/or government units, etc. The aforementioned ratios of 80%, 20%, etc. are merely examples, and they are not limit the embodiments of the present invention.

TABLE 2

| | | |
|---|---|---|
| the sample | the sample number for | |
| number for | the second-level first | |
| the | learning model M21 + the | |
| first-level | sample number for the | the sample |
| learning | second-level second | number for |
| model M1 | learning model M22 | verification |

100%

| 80% | | 20% |
|---|---|---|
| 80% | 20% | — |

After obtaining the first-level learning model M1, the second-level first learning model M21 and the second-level second learning model M22, a predicted cancer type of at least one to-be-tested sample could be obtained by using there learning models, and further examples are described below.

Referring to FIG. 2, FIG. 2 shows a functional block diagram of a cancer type prediction system 200 according to an embodiment of the present invention. The cancer type prediction system 200 includes a storage unit 210 and a prediction unit 220. The storage unit 210 and the prediction unit 220 are, for example, physical circuits formed by at least one semiconductor manufacturing process. Specifically, the storage unit 210 may be a memory, which may be integrated into the prediction unit 220 or disposed separately from the prediction unit 220. In an embodiment, the prediction unit 220 and/or the storage unit 210 could be integrated in a controller or a processor.

The storage unit 210 is configured to store the first-level learning model M1 and the second-level first learning model M21. The prediction unit 220 is configured to: (1). obtain a first prediction cancer type C1_{PT} of the to-be-tested sample P by inputting a sample CNV VPT and a sample gender SPT of a to-be-tested sample PT to the first-level learning model M1; (2). determine whether the sample gender SPT of the to-be-tested sample PT is the second gender S_{P2}; and (3). when the sample gender of the to-be-tested sample PT is the second gender S_{P2}, obtain a second prediction cancer type C2_{PT} of the to-be-tested sample PT by inputting the first prediction cancer type C1_{PT} of the to-be-tested sample PT to the second-level first learning model M21.

The aforementioned sample gender SPT is not limited to male or female. In other words, the sample gender SPT could be male or female.

In addition, the storage unit 210 is further configured for storing the second-level second learning model M22. The prediction unit 220 is further configured to: (1). determine whether the sample gender SPT of the to-be-tested sample PT is the third gender S_{P3}; and, (2). when the sample gender SPT of the to-be-tested sample PT is the third gender S_{P3}, obtain the second prediction cancer type C2_{PT} of the to-be-tested sample PT by inputting the first prediction cancer type C1_{PT} of the to-be-tested sample PT to the second-level second learning model M22.

The accuracy of the second prediction cancer types C2_{PT} of several to-be-tested samples PT is described below. The following tables 3-1 and 3-2 list the data analysis of the second prediction cancer types C2_{PT} and the actual suffering cancer types of several to-be-tested samples, such as sensitivity Sens, specificity Spec, positive predictive value (PPV) and negative predictive value (NPV). For esophageal cancer, due to its location (just located between the head/neck and the stomach) being special, the prediction could be regarded as a correct result as long as the prediction result is the head and neck cancer or the gastrointestinal cancer.

TABLE 3-1

| | test results for male | | | | | | | |
|---|---|---|---|---|---|---|---|---|
| | Sens | | Spec | | PPV | | NPV | |
| category | T1 | T3 | T1 | T3 | T1 | T3 | T1 | T3 |
| health | 1 | 1 | 0.93 | 0.93 | 0.68 | 0.68 | 1 | 1 |
| anterior mediastinum cancer | 0.19 | 0.44 | 0.99 | 0.99 | 0.45 | 0.67 | 0.97 | 0.98 |
| Gastrointestinal cancer | 0.68 | 0.92 | 0.96 | 0.96 | 0.71 | 0.77 | 0.95 | 0.99 |
| kidney cancer | 0.83 | 0.88 | 0.97 | 0.97 | 0.78 | 0.79 | 0.98 | 0.98 |
| liver cancer | 0.66 | 0.9 | 0.99 | 0.99 | 0.87 | 0.9 | 0.98 | 0.99 |
| lung cancer | 0.74 | 0.89 | 0.93 | 0.93 | 0.59 | 0.63 | 0.96 | 0.98 |
| male cancer | 0.81 | 0.96 | 0.97 | 0.97 | 0.78 | 0.81 | 0.97 | 0.99 |
| brain cancer | 0.87 | 0.96 | 0.99 | 0.99 | 0.91 | 0.92 | 0.98 | 0.99 |
| breast cancer | 0 | 0.5 | 1 | 1 | Null | 1 | 1 | 1 |
| head and neck cancer | 0.49 | 0.85 | 0.97 | 0.97 | 0.55 | 0.68 | 0.96 | 0.99 |
| Bladder cancer | 0.65 | 0.74 | 1 | 1 | 0.97 | 0.97 | 0.98 | 0.98 |
| female cancer | Null | Null | 1 | 1 | Null | Null | 1 | 1 |
| esophageal cancer | 0.83 | 0.92 | 1 | 1 | 1 | 1 | 0.99 | 1 |

TABLE 3-2

| | test results for female | | | | | | | |
|---|---|---|---|---|---|---|---|---|
| | Sens | | Spec | | PPV | | NPV | |
| category | T1 | T3 | T1 | T3 | T1 | T3 | T1 | T3 |
| health | 1 | 1 | 0.91 | 0.91 | 0.5 | 0.5 | 1 | 1 |
| anterior mediastinum cancer | 0.16 | 0.89 | 0.99 | 0.99 | 0.68 | 0.92 | 0.94 | 0.99 |
| Gastrointestinal cancer | 0.77 | 0.91 | 0.97 | 0.97 | 0.72 | 0.75 | 0.98 | 0.99 |
| kidney cancer | 0.61 | 0.74 | 0.99 | 0.99 | 0.77 | 0.8 | 0.98 | 0.99 |
| liver cancer | 0.53 | 0.62 | 1 | 1 | 0.75 | 0.78 | 0.99 | 0.99 |
| lung cancer | 0.57 | 0.85 | 0.96 | 0.96 | 0.56 | 0.65 | 0.96 | 0.99 |
| male cancer | Null | Null | 1 | 1 | Null | Null | 1 | 1 |
| brain cancer | 0.9 | 0.91 | 0.99 | 0.99 | 0.92 | 0.93 | 0.98 | 0.99 |
| breast cancer | 0.79 | 0.95 | 0.96 | 0.96 | 0.82 | 0.85 | 0.95 | 0.99 |
| head and neck cancer | 0.32 | 0.74 | 1 | 1 | 0.69 | 0.83 | 0.98 | 0.99 |
| Bladder cancer | 0.23 | 0.38 | 1 | 1 | 0.75 | 0.83 | 0.98 | 0.99 |
| female cancer | 0.8 | 0.98 | 0.89 | 0.89 | 0.73 | 0.77 | 0.93 | 0.99 |
| esophageal cancer | 0.43 | 0.43 | 1 | 1 | 1 | 1 | 0.99 | 1 |

The sensitivity Sens, the specificity Spec, the positive predictive value PPV and the negative predictive value NPV are represented by the following formulas (1) to (4) respectively, wherein the definitions of parameters TP, FP, FN and TN are shown in Table 4.

$$Sens = TP / ( TP + FN ) \qquad ( 1 )$$

$$Spec = TN / ( FP + TN ) \qquad ( 2 )$$

$$PPV = TP / ( TP + FP ) \qquad ( 3 )$$

$$NPV = TN / ( FN + TN ) \qquad ( 4 )$$

TABLE 4

actual situation

| prediction | suffering from | not suffering from |
|---|---|---|
| result | the cancer | the cancer |
| Positive | the prediction | the prediction |
| | result is A | result is A |
| | cancer, and the | cancer, but the |
| | actual situation | actual situation |
| | also is A cancer | is not A cancer |
| | (true positive, | (false positive, |
| | TP) | FP) |
| Negative | the prediction | the prediction |
| | result is not A | result is not A |
| | cancer, but the | cancer, and the |
| | actual situation | actual situation |
| | is A cancer | is not cancer |
| | (false negative, | (true negative, |
| | FN) | TN) |

In addition, T1 (Top 1) in the above tables 3-1 and 3-2 refers to: in terms of TP, when the actual situation is A cancer and the cancer having the highest probability in the prediction result is A cancer, then the value of TP adds by 1; when the actual situation is A cancer but the cancer having the highest probability in the prediction result is not A cancer, then the value of TP adds by 0. T3 (Top 3) refers to: in terms of TP, when the actual situation is A cancer and the top 3 with the highest probability in the prediction results also include A cancer, then the value of TP adds by 1; when the actual situation is A cancer but the top 3 with the highest probability in the prediction results do not include A cancer, then the value of TP adds by 0.

According to the analysis data in Table 3-1 and 3-2, for some cancers (for example, gastrointestinal cancer, liver cancer, male cancer, brain cancer, breast cancer, female cancer, etc.), the sensitivity Sens is higher than 90%. In early screening applications, it is recommended that the sensitivity is placed in the highest priority.

As shown in Table 5 below, for the first-level learning model M1, the accuracy of predicting male cancer is 68.5%, while the accuracy of predicting female cancer is 69.8%. For the second-level learning model, the accuracy of predicting male cancer increases to 72.91%, while the accuracy in predicting female cancer increases to 71.21%.

TABLE 5

| | first-level learning | second-level learning |
|---|---|---|
| gender | model M1 | model |
| male | 68.5% | 72.91% |
| female | 69.8% | 71.21% |

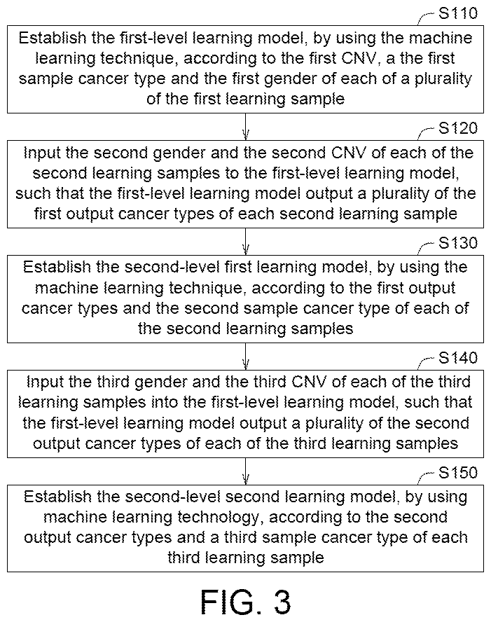

Referring to FIG. 3, FIG. 3 shows a flowchart of an establishing method for a cancer type prediction model in the cancer type prediction model establishment system 100 in FIG. 1.

In step S110, referring to FIG. 1, the first-level learning model establishment unit 110 establishes the first-level learning model M1, by using the machine learning technique, according to the first CNV V_{P1}, a the first sample cancer type C1_{P1} and the first gender S_{P1} of each of a plurality of the first learning sample P1. In an embodiment, as shown in FIG. 1, the first-level learning model establishment unit 110 is further configured to: establish the first-level learning model M1, by using machine learning technology, according to the fourth CNV V_{P4}, the health category H_{P4} and the fourth gender SP4 of each of a plurality of the healthy samples P4 (not illustrated).

In step S120, as shown in FIG. 1, the second gender S_{P2} and the second CNV V_{P2} of each of the second learning samples P2 are inputted to the first-level learning model M1, the first-level learning model M1 outputs a plurality of the first output cancer types C2_{P2} of each second learning sample P2.

In step S130, as shown in FIG. 1, the second-level first learning model establishment unit 120 establishes the second-level first learning model M21, by using the machine learning technique, according to the first output cancer types C2_{P2} and the second sample cancer type C1_{P2} of each of the second learning samples P2. In an embodiment, the second-level first learning model establishment unit 120 could further establish the second-level first learning model M21 according to the age G_{P2} of each second learning sample P2.

In step S140, referring to FIG. 1, the first-level learning model establishment unit 110 inputs the third gender S_{P3} and the third CNV V_{P3} of each of the third learning samples P3 to the first-level learning model M1, such that the first-level learning model M1 outputs the corresponding second output cancer types C2_{P3}.

In step S150, as shown in FIG. 1, the second-level second learning model establishment unit 130 establishes the second-level second learning model M22, by using machine learning technology, according to the second output cancer types C2_{P3} and a third sample cancer type C1_{P3} of each third learning sample P3. In an embodiment, the second-level second learning model establishment unit 130 could further establish the second-level second learning model M22 according to the age Gp3 of each third learning sample P3.

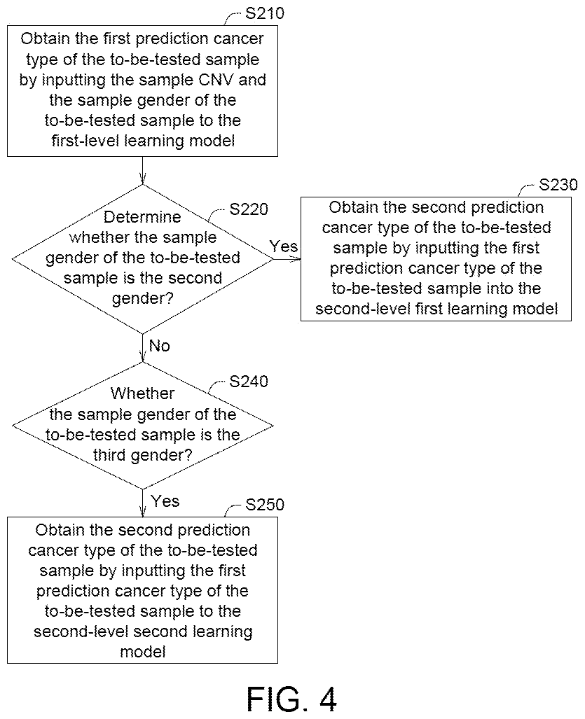

Referring to FIG. 4, FIG. 4 shows a flow chart of the cancer type prediction method of the cancer type prediction system 200 in FIG. 2.

In step S210, as shown in FIG. 1, the prediction unit 220 obtain the first prediction cancer type C1_{PT} of the to-be-tested sample PT by inputting the sample CNV V_{PT} and the sample gender S_{PT} of the to-be-tested sample PT to the first-level learning model M1.

In step S220, referring to FIG. 1, the prediction unit 220 determines whether the sample gender S_{PT} of the to-be-tested sample PT is the second gender S_{P2}. If the sample gender S_{PT} of the to-be-tested sample PT is the second gender S_{P2}, the process proceeds to step S230; if not, the process proceeds to step S240.

In step S230, as shown in FIG. 1, the prediction unit 220 obtains the second prediction cancer type C2_{PT} of the to-be-tested sample PT by inputting the first prediction cancer type C1_{PT} of the to-be-tested sample PT to the second-level first learning model M21.

In step S240, the predicting unit 220 determines whether the sample gender S_{PT} of the to-be-tested sample PT is the third gender S_{P3}. If the third gender S_{P} of the to-be-tested sample PT is the third gender S_{P3}, the process proceeds to step S250.

In step S250, the prediction unit 220 obtains the second prediction cancer type C2_{PT} of the to-be-tested sample PT by inputting the first prediction cancer type C1_{PT} of the to-be-tested sample PT to the second-level second learning model M22.

In the present embodiment, the sample gender S_{PT} is either the second gender S_{P2} or the third gender S_{P3}, and thus the process could also omit step S240.

To sum up, an embodiment of the present disclosure discloses a cancer type prediction model establishment system, a cancer type prediction system and a cancer type prediction method using the same. The cancer type prediction model establishment system establishes the two level learning models by using the machine learning technology. The cancer type prediction system could easily and quickly predict the cancer type of the to-be-tested sample by using such two level learning models. In an embodiment, the first-level learning model is, for example, a mixed-type learning model (for example, not limited to gender), accordingly it could avoid the learning (or training) error caused by some cancer types with a small number of samples. The second-level learning model could predict the cancer type with a specific gender.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

1. A cancer type prediction system, comprising:
a first-level learning model establishment unit configured to:
establish a first-level learning model, by using a machine learning technique, according to a first CNV (copy number variation), a first sample cancer type and a first gender of each of a plurality of first learning sample;
use a second gender and a second CNV of each of a plurality of second learning sample as an input of the first-level learning model, so that the first-level learning model outputs a plurality of first output cancer types of each second learning sample; and
a second-level first learning model establishment unit configured to:
establish a second-level first learning model, by using the machine learning technique, according to the first output cancer types and a second sample cancer type of each of the second learning samples.

2. The cancer type prediction system as claimed in claim 1, wherein the first genders of these persons to whom the first learning samples belongs are a combination of male and female, and the second genders of these persons to whom all of the second learning samples belongs are male or female.

3. The cancer type prediction system as claimed in claim 1, wherein the first-level learning model establishment unit further configured to:
input a third gender and a third CNV of each of a plurality of third learning samples to the first-level learning model, so that the first-level learning model outputs a plurality of second output cancer types of each of the third learning samples;
wherein the cancer type prediction system further comprises:
a second-level second learning model establishment unit configured to:
establish a second-level second learning model, by using machine learning technology, according to the second output cancer types and a third sample cancer type of each third learning sample;
wherein the third genders of these persons to whom all of the second learning samples belongs are male or female.

4. The cancer type prediction system as claimed in claim 3, wherein the second-level second learning model establishment unit further configured to:
establish the second-level second learning model according to an age of each third learning sample;
wherein the second-level first learning model establishment unit further configured to:
establish the second-level first learning model according to an age of each second learning sample.

5. A cancer type prediction system, comprising:
a storage unit configured to:
store the first-level learning model and the second-level first learning model as claimed in claim 1;
a prediction unit configured to:
obtain a plurality of first prediction cancer types of the to-be-tested sample by inputting a sample CNV and a sample gender of a to-be-tested sample to the first-level learning model;
determine whether a sample gender of the to-be-tested sample is the second gender; and
when the sample gender of the to-be-tested sample is the second gender, obtain a second prediction cancer type of the to-be-tested sample by inputting the first prediction cancer type of the to-be-tested sample to the second-level first learning model.

6. The cancer type prediction system as claimed in claim 5, wherein the storage unit further configured to store a second-level second learning model, and the prediction unit configured to:
determine whether the sample gender of the to-be-tested sample is a third gender; and
when the sample gender of the to-be-tested sample is the third gender, obtain the second prediction cancer type of the to-be-tested sample by inputting the first prediction cancer type of the to-be-tested sample to the second-level second learning model.

7. An establishing method for a cancer type prediction model, comprising:
establishing a first-level learning model, by using a machine learning technique, according to a first CNV, a first sample cancer type and a first gender of each of a plurality of first learning sample;
using a second gender and a second CNV of each of a plurality of second learning sample as an input of the first-level learning model, so that the first-level learning model outputs a plurality of first output cancer types of each second learning sample; and
establishing a second-level first learning model, by using the machine learning technique, according to the first output cancer types and a second sample cancer type of each of the second learning samples.

8. The establishing method as claimed in claim 7, further comprising:
inputting a third gender and a third CNV of each of a plurality of third learning samples to the first-level learning model, so that the first-level learning model output a plurality of second output cancer types of each of the third learning samples;
establishing a second-level second learning model, by using machine learning technology, according to the second output cancer types and a third sample cancer type of each third learning sample;
wherein the second gender is different from the third gender.

9. The establishing method as claimed in claim 8, wherein establishing the second-level second learning mode according to the second output cancer types and a third sample cancer type of each third learning sample further comprises:
establishing the second-level second learning model according to an age of each third learning sample;
wherein establishing the second-level first learning model according to the first output cancer types and the second sample cancer type of each of the second learning samples further comprises:
establishing the second-level first learning model according to an age of each second learning sample.

10. A cancer type prediction method, comprising:
obtaining a first prediction cancer type of the to-be-tested sample by inputting a sample CNV and a sample gender of a to-be-tested sample to a first-level learning model as claimed in claim 1;
determining whether a sample gender of the to-be-tested sample is the second gender; and
when the sample gender of the to-be-tested sample is the second gender, obtaining a second prediction cancer type of the to-be-tested sample by inputting the first prediction cancer type of the to-be-tested sample to the second-level first learning model as claimed in claim 1.
